(19) United States
(12) Patent Application Publication
(10) Pub. No.: US 2004/0147934 A1
(43) Pub. Date: Jul. 29, 2004

(54) Oscillating, steerable, surgical burring tool and method of using the same

(76) Inventor: P. Douglas Kiester, Irvine, CA (US)

Correspondence Address: Daniel L. Dawes, MYERS DAWES ANDRAS & SHERMAN LLP, 11th Floor, 19900 MacArthur Boulevard, Irvine, CA, 92612, US

(21) Appl. No.: 10/689,014

(22) Filed: Oct. 20, 2003

Related U.S. Application Data
(60) Provisional application No. 60/419,803, filed on Oct. 18, 2002.

(51) Int. Cl. A61B 17/32; A61B 17/00

(52) U.S. Cl. 606/80; 606/171; 606/180

(57) ABSTRACT

The invention is an oscillating, high speed burring instrument comprised of a handpiece, an elongate arthroscopic catheter extending distally from handpiece and terminating in a flexible or hinged portion which itself terminates with an oscillating burr. At least the distal portion of torsional drive shaft is radially flexible to accommodate the flexibility of the flexible or hinged portion of the catheter. A high speed oscillation of the burr is employed effective for cutting or abrading bone, which is typically oscillated at 10 kHz or higher. The burr is oscillated over a substantial arc, namely a majority portion of a full circle. The burr is not shielded in any manner and is fully exposed to the operational theater. The burr cuts or abrades bone or hard matter, while leaving softer tissues substantially or entirely undamaged.

### RELATED APPLICATIONS

The present application is related to U.S. Provisional Patent Application serial No. 60/419,803, filed on Oct. 18, 2002, which is incorporated herein by reference and to which priority is claimed pursuant to 35 USC 119.

### BACKGROUND OF THE INVENTION

1. Field of the Invention

The invention relates to the field of surgical tools and methods, and in particular to minimally invasive cutting tools and methods, which differentially cut one tissue type in preference to others.

2. Description of the Prior Art

Generally in the field of surgery, arthroscopic cutting instruments have encountered numerous problems, the primary one being that the instruments clog or jam from tissue buildup or cut indiscriminately all tissue types which come into contact with the cutting tool. The tissue jamming or clogging requires frequent cleaning or substitution of the prior art instruments which is not only time consuming thus increasing the time of the procedure as well as decreasing the number of procedures possible in a given operating room facility but also contributes to physician fatigue thus increasing the chances of error.

The continuing emphasis of minimally invasive arthroscopic surgery also means that the surgeon has less opportunity to view the field of operation and to control what tissues may be contacted by the cutting tool. Therefore, if the cutting tool cuts all tissue types without discrimination, such as bone and soft or fibrous tissues, then full and uninterrupted observation and controllability of the cutting tool is an absolute prerequisite to its use. The greater degree of observability which is demanded, the greater is the degree of invasion since the field of operation must either be cut opened to view or must be enlarged to allow the insertion of observation instruments and means for clearing them. Further, even when optical fibers can be used as scopes and kept clear, a flat two dimensional image is provided which does not allow the physician easy assessment of the three dimensional positioning of the tool relative to nearby tissues. Hence, a high degree of skill and care is required in order to selectively use a cutting instrument. The problem is exacerbated when bone is to be cut away, since high speed cutting instruments are required. Still further, when the spinal column is the subject of operation, the proximity of bone to critical neurological tissue renders to the use of arthroscopic cutting instruments for orthopedic spinal surgical in a minimally invasive manner virtually unobtainable.

The following patents constitute representative types of prior art instrumentation directed toward soft tissue removal. The invention is distinguished in that it is capable of removing bone. The prior art is replete with numerous arthroscopic surgical instruments utilizing a cutting tube mounted within an outer cutting housing, the inner cutter member being hollow and connected to a source of suction. These cutting tubes either rotate or reciprocate within the outer tube housing. Examples of such cutting instruments are shown in U.S. Pat. No. 5,324,301, issued on Jun. 28, 1994, and U.S. Pat. No. 5,286,253, issued on Feb. 15, 1994, the latter showing a similar apparatus with a toothed rotating cutter. Another U.S. Pat. No. 4,274,414, issued on Jun. 23, 1981, discloses an arthroscopic cutter having a coupling member with a central chamber which deflects fluid and tissues cut by the cutter into a cutter tube to a suction box. Another arthroscopic surgery instrument with a blunt cutter tip and similar construction is shown in U.S. Pat. No. 4,203,444, issued on May 20, 1990. A variety of cutter tips which can be used with arthroscopic surgical instruments are shown in U.S. Pat. No. 4,705,038, issued on Nov. 10, 1987, which patent also shows a suction sQurce which extends from the cutter tube through the instrument body exiting out the rear. A cutting lipectomy device is shown in U.S. Pat. No. 4,815,462 issued on Mar. 28, 1989.

U.S. Pat. No. 5,403,276 issued Apr. 4, 1995 is directed toward a combined tissue removal system which uses a reciprocating cutting blade and feedback control for aspiration and irrigation circuits used in the system.

Attempts to overcome clogging and jamming of these types of instruments due to collection of tissue and other materials which have been severed from the body during cutting while performing the surgical procedure has been to attempt to remove these materials so that they will not have a chance to collect in the instrument or pausing during surgery and breaking down and cleaning the instruments. Unfortunately, the cleaning of these instruments can be difficult and time consuming in a surgical environment. U.S. Pat. No. 4,108,182, issued on Aug. 22, 1978, shows a surgical instrument with a removable cutter head. The cutter head is provided with a single lumen exterior conduit leading either to the suction or the fluid source so that fluid or suction can alternately be provided along the single lumen flexible tube to the hollow cutting tube. U.S. Pat. No. 5,059,204 issued on Oct. 22, 1991 discloses an ocular guillotine cutter placed within a swagged outer needle.

Prior art attempts to provide discriminatory cutting tools have generally been directed to cutters which are shielded in some manner to prevent tissue from contact the cutting tool surface from an undesired direction. This typically results in a limited amount of the cutting tool surface being presented and thus limits the cutting efficiency and manipulability of the tool.

In spine surgery, frequently the goal is to remove bone, which has overgrown into the spinal canal without damaging the delicate neural elements or creating a leak in the balloon-thin water-containing dura. In this age of decreasingly invasive surgical instruments and techniques, even in endoscopic surgical approaches the visualization of the bone being removed is frequently difficult. For example, even with a large open procedure, removing overgrown bone from the exiting nerve root's neuroforemina is very difficult, and dangerous to the nerve root. To be able to remove hard bone without danger of damage to nearby soft tissues is one of the objects of the invention.

What is needed is some type of surgical cutting tool, which automatically discriminates between tissue types so that it will efficiently cut bone, hard tissues or matter, but will not cut surrounding softer tissues.

### BRIEF SUMMARY OF THE INVENTION

The invention is an oscillating, high speed burring instrument comprised of a handpiece, an elongate arthroscopic catheter extending distally from handpiece and terminating in a flexible or hinged portion which itself terminates with an oscillating burr. An electrical motor is connected via a torsional drive shaft, which is axially disposed in the catheter and connected to the burr. At least the distal portion of torsional drive shaft is radially flexible to accommodate the flexibility of the flexible or hinged portion of the catheter. By whatever means can be employed to oscillate burr, a high speed oscillation is employed effective for cutting or abrading bone, which is typically oscillated at 10 kHz or higher.

The burr is oscillated over a substantial arc, namely a majority portion of a full circle. The burr is not shielded in any manner and is fully exposed to the operational theater. Thus, in the illustrated embodiment access to the burr is substantially unimpeded so that cutting in virtually all directions is possible and cooling, and clearing by fluid irrigation and fluid and debris removal by suction can be performed without hindrance.

Regardless of the means by which the burr is oscillated, it is a feature of the invention that the burr cuts or abrades bone or hard matter, while leaving softer tissues substantially or entirely undamaged.

In one embodiment the drive shaft is connected to the burr by a biased slip clutch assembly. A driving shaft is connected to a rotary electric motor. The primary engagement of driven shaft to driving shaft is by means of frictional engagement with each other in a section of interleaved hubs where segmental cylindrical portions of each are telescopically overlapped. As the drive shaft rotates it winds a torsional spring which begins to build up a resisting torque. At some point the degree of torsion applied to the spring will generate a resisting torque applied to driven shaft, which will equal and then exceed the force of the frictional engagement between driving shaft and driven shaft. At this point the frictional engagement between driving shaft and driven shaft drops and the stored torsional energy in the drive shaft breaks driven shaft free from driving shaft. The driven shaft returns by the spring force back to an initial angular position. The driving shaft and driven shaft will rotate relative to each other until their segmental cylindrical portions again begin to overlap. When the overlap is sufficient, the frictional engagement causes the driving shaft to once again grab the driven shaft and they once again rotated together. The process continues with the result that the drive shaft rotationally oscillates at a frequency synchronized to the rotation of driving shaft. The burr is connected to the drive shaft and is thus oscillated at the same frequency through a fractional arc of a circle.

In other embodiments the drive shaft assembly comprises a segmental gear-pulley combination with a belt, an eccentric pin-crank combination, a bibbed or multilobed counter-rotating gear combination, or a multiple cam combination.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a simplified diagram of the instrument of the invention illustrating the selectively bending of the steerable burring tool.

FIG. 2 is a simplified diagram of the flexible terminal portion of the catheter connected to the burring tool of FIG. 1, which portion is structured to bend in only one plane.

FIG. 3 is a simplified exploded perspective view of one embodiment of a mechanism for converting full rotary motion into oscillating rotary motion.

FIG. 4 is a diagrammatic perspective view of an alternative embodiment of a belt driven, segmental gear or pulley transmission for rotation-to-oscillation conversion.

FIGS. 5a-5c is an embodiment of the belt driven transmission of FIG. 4 showing various configurations of the drive and driven pulley-gears during the cycle.

FIGS. 6a-6d are diagrammatic end views of an embodiment of the transmission for conversion of rotation to oscillation using an eccentric pin-crank combination.

FIG. 7 is a diagrammatic top plan view of the transmission of FIGS. 6a-6d where a gear has been added for elimination of offset between the axes of rotation and oscillation and for design variation of the angular magnitude of oscillation.

FIGS. 8a-8c includes a diagrammatic top plan view of another embodiment of the transmission for conversion of rotation to oscillation using counter-rotating bibbed segmental gears.

FIGS. 9a-9d is a simplified end view of an embodiment of the transmission for conversion of rotation to oscillation using a cam combination.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF TH PREFERRED EMBODIMENTS

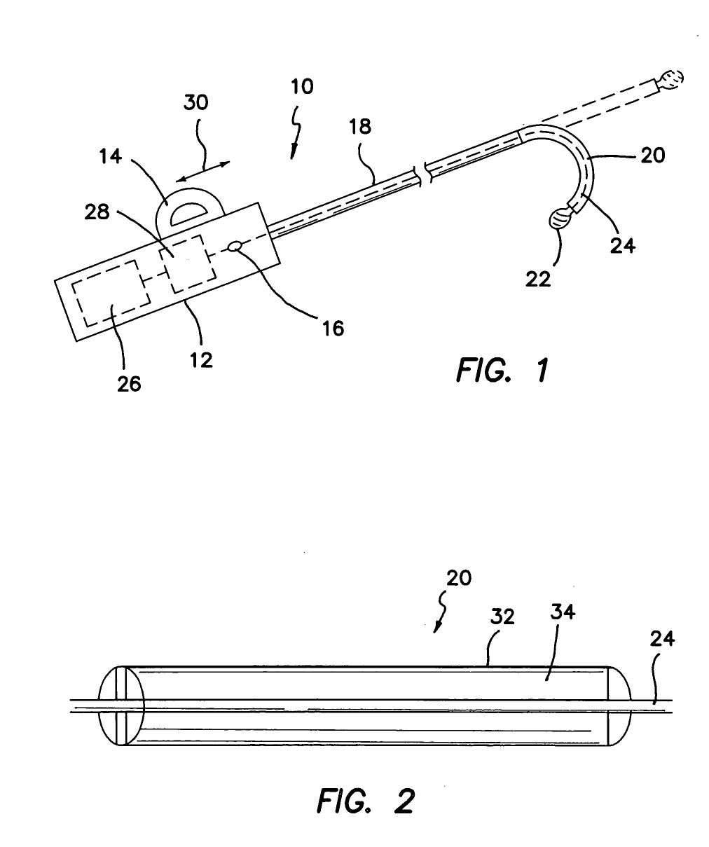

FIG. 1 is a simplified diagrammatic side view of an oscillating, high speed burring instrument, generally denoted by reference numeral 10. Instrument 10 is illustratively comprised of a handpiece 12 held proximally by the surgeon, which handpiece 12 includes the controls, motor and portable power source for instrument 10. Connection to an external fixed power source, such as conventional electrical lines, is also contemplated. An elongate arthroscopic catheter 18 extends distally from handpiece 12 and terminates in a flexible or hinged portion 20 which itself terminates with an oscillating burr 22. Handpiece 12 is also provided with a finger grip 14 used to control the curvature of flexible or hinged portion 20 by means of a conventional tension wire or other equivalent means (not shown) and a start/stop button 16. Variable speed controls may also be provided if desired.

A rechargeable battery 26 is included within handpiece 12 as a power source, or external power may be provided. An electrical motor 28 is included in handpiece 12 and connected via a torsional drive shaft 24 which is axially disposed in catheter 18 and connected to burr 22. In general, motor 28 which serves as the motive source for burr 22 may be an electrical rotary motor, but includes other motive means or sources such as oscillating motors or solenoids. The torsional drive shaft 24 may be radially flexible in whole or part, while being sufficiently torsionally stiff to provide effective rotational drive to burr 22. At least the distal portion of torsional drive shaft 24 is radially flexible to accommodate the flexibility of flexible or hinged portion 20 of catheter 18.

Electrical motor 28 may be a conventional oscillating motor or a conventional rotary motor coupled to a mechanical converter, which translates the rotational output of motor 28 into an oscillatory rotational motion. Many types of motive mechanisms exist by which an oscillatory motion can be imparted to drive shaft 24 and hence to burr 22. The means described below for imparting oscillatory motion to burr 22 is only one example, and the invention must be understood wherever appropriate to be generally directed to any type of motive mechanism now known or later devised capable of imparting oscillating motion to burr 22, including micromotors which may be mounted in the distal end of catheter 18 itself near burr 22. Thus, hydraulic, ultrasonic, electromagnetic and piezoelectric drives as examples are contemplated within the scope of the invention as well as electrical motive devices.

By whatever means can be employed to oscillate burr 22, a high speed or rate of oscillation is employed effective for cutting or abrading bone or hard tissue such as cartilage, which burr 22 is typically oscillated at a rate of 10 kHz or higher.

In the illustrated embodiment of the invention burr 22 is oscillated over a substantial arc, namely a majority portion of a full circle. For example, burr 22 is rotated from a positioned denoted as 0° to 270° and back again in a single cycle. Lesser arcs of oscillation may be employed and controlled through conventional motor controls connected to motor 28 if desired. The size of the arc which may be used in the oscillation can be varied according to the nature of burr 22, the size, sharpness and design of the cutting edges as well as the nature of the intended material to be cut or abraded.

Burr 22 is generally not shielded in any manner and is fully exposed to the operational theater, or at least is minimally shielded or occluded. For example, burr 22 may be provided in the form of a spherical or ball burring head having a cutting or abrading surface on nearly the full spherical surface, except for the area connected to drive shaft 24. It is further to be expressly understood that the cutting or abrading surface of burr 22 may have any form now known or later devised and may be interchangeably connected to drive shaft 24 to allow the use of a multiple of different oscillating tools and burrs in instrument 10. Thus, in the illustrated embodiment access to burr 22 is substantially unimpeded so that cutting in virtually all directions is possible and cooling, and clearing by fluid irrigation and fluid and debris removal by suction can be performed without hindrance. Instrument 10 can thus be combined with various irrigation and suction systems or mechanisms as desired without departing from the scope of the invention. While the cutting implement is termed a “burr” other types of cutting, abrading, or grinding tools, drills, knives or workpieces can be equally substituted.

Regardless of the means by which burr 22 is oscillated, it is a feature of the invention that burr 22 cuts or abrades bone or hard matter, while leaving softer tissues substantially or entirely undamaged. While it is not completely understood why an oscillating burr discriminatingly cuts or abrades bone and not soft tissue, its operation is believed to be as follows. Since burr 22 generally does not rotate more than a single full revolution before reversing, whatever the cutting or abrading edge on the surface of burr 22 may be, it does not cut into or substantially abrade the softer tissue. In one sense the cutting or abrading edge on the surface of burr 22 bumps along the surface of the softer tissue or may deform it, but does not or is unable to cut into it or grab it sufficiently to tear it before burr stops and reverses to release the softer tissue. Bone or hard matter on the other hand is unyielding and will not move or rotate with burr 22 and then release from burr 22 with its oscillation so that cutting or abrading occurs. The cutting or abrading action of burr 22 is believed to be a function of the size of the arc through which it oscillates, the rate of oscillation, the nature of the cutting or abrading surface of burr 22, and the nature of the tissue in contact with the cutting or abrading surface. Each of these factors can be adjusted according to the invention in order to realize the discriminatory cutting action of burr 22 relative to hard and soft tissues. In the illustrated embodiment a conventional surgical burring tool oscillated at high speed, i.e. approximately 5 kHz or higher, through an arc of more than approximately 180° will cut or abrade vertebra, but not the softer dura adjacent to it.

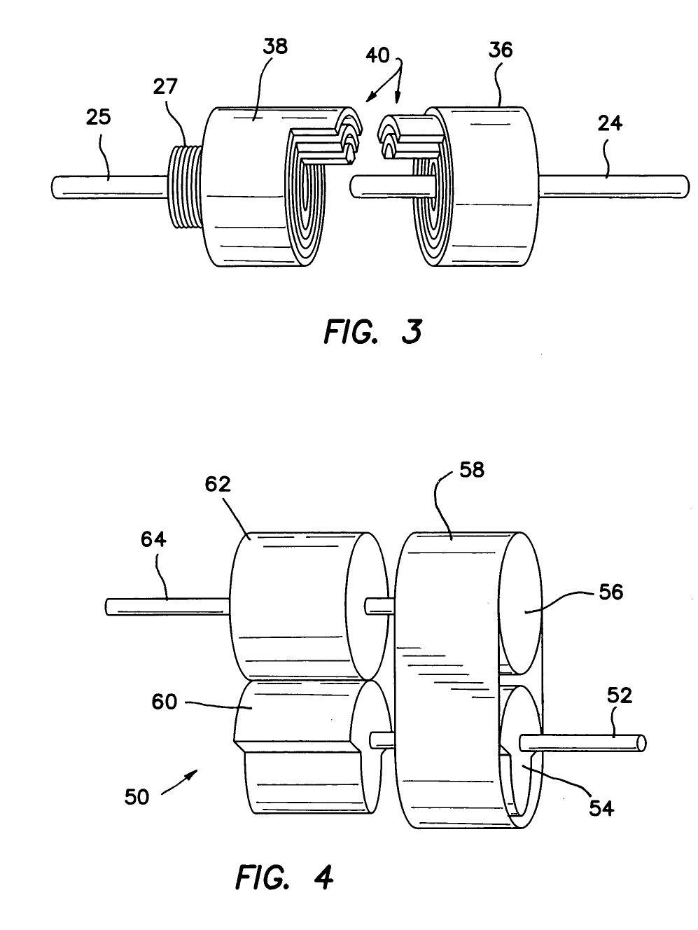

One mechanism for providing a mechanical converter between rotary motion and oscillatory motion is shown in simplified exploded side perspective view in FIG. 3. Drive shaft 24 in this embodiment is coupled to an outer driving hub 36 and is connected to a rotary electric motor 28. Drive shaft 24 is fixed to driving hub 36. A driven hub 38 is telescopically coupled to driving hub 36 and frictionally engaged therewith. The primary engagement of driven hub 38 to driving hub 36 is by means of frictional engagement with each other by one or more interleaved arcs 40 where they are telescopically overlapped. Shaft 24 may be extended through or into a bore (not shown) defined in driving hub 36 into driven hub 38 for stability and centering, but without significant frictional engagement. The engaged surfaces of driving hub 36 and driven hub 38 may be treated, coated, inlaid or otherwise modified to create the desired frictional engagement and longevity.

When the segmental cylindrical surfaces of driving hub 36 and driven hub 38 are aligned, they are maximally engaged with each other and rotation of driving hub 36 will rotate driven hub 38. However, oscillating shaft 25, is fixed to driven hub 38 as is coil torsion spring 27. As oscillating shaft 25 rotates it winds torsional spring 27 which has one end fixed relative to shaft 25 and hub 38, and it begins to build up a resisting torque. At some point the degree of torque applied to oscillating shaft 25 will generate a resisting torque applied to driven hub 38, which will equal and then exceed the force of the frictional engagement between driving hub 36 and driven hub 38. At this point driven hub 38 will begin to lag behind driving hub 36 causing their overlapping interleaved portions 40 to rotate relative to each other. The degree of overlap of the segmental arcs or cylinders 40 will decrease until it becomes minimal or zero. When the frictional engagement between driving hub 36 and driven hub 38 drops below a threshold value, the stored torsional energy in spring 27 will break driven hub 38 free from driving hub 36 and rotate driven hub 38 and oscillating shaft 25 back to an initial angular position. Oscillating shaft 25 will thus stop and reverse its rotation, since it is connected to driven hub 38. Meanwhile driving hub 36 continues to rotate in the same direction as it was initially rotating. The torsional energy stored in spring 27 will be dissipated and driving hub 36 and driven hub 38 will rotate relative to each other until their segmental cylindrical portions 40 again begin to overlap. When the overlap is sufficient, the frictional engagement causes driving hub 36 to once again grab driven hub 38 when it overcomes the torsional resistance of spring 27, and to begin to turn driven hub 38 and oscillating shaft 25 back in the original direction. The process continues with the result that oscillating shaft 25 rotationally oscillates at a frequency synchronized to the rotation of driving shaft 24. Burr 22, connected to oscillating shaft 25 is thus oscillated at the same frequency through a fractional arc. It is to be expressly understood that many mechanical and other means can be used to convert full rotary motion into oscillating rotary motion and that the invention is not necessarily limited to the disclosed embodiment. The combination of FIG. 3 can be defined as a biased slip clutch assembly or combination.

Flexible or hinged portion 20 is a resilient or at least flexible portion or extension of catheter 18 and may be comprised of a resilient tube or coil spring forming at least part of an outer cylindrical housing in which drift shaft 24 is disposed. A tension wire (not shown) is connected at its distal end to a distal end or portion of flexible or hinged portion 20 and at its proximal end to finger grip 24. Movement by the surgeon of finger grip 24 in the directions of arrow 30 thus causes the tension wire connected to flexible or hinged portion 20 to be drawn proximally or released distally to cause flexible or hinged portion 20 to curve back toward handpiece 12 as shown in solid outline in FIG. 1 or to straighten as shown in dotted outline in FIG. 1.

In the preferred embodiment flexible or hinged portion 20 is restrained to bend in a single plane in order to avoid wobbling out of plane with burr 22 is oscillated and forced against bone tissue. Such restraint can be achieved by comprising portion 20 out of hinged metal links which are rotatably connected to each other by pins which allow rotation about a single axis, or by inserting a flat stiffening ribbon or leaf spring into portion 20, which is flexible in one direction and stiff in the transverse direction. For example, as diagrammatically depicted in FIG. 2, portion 20 may be comprised of a flexible polymeric tube 32 through which a tight coil spring drift shaft 24 is axially disposed and in which tube 32 a flat spring strip 34 is disposed diametrically or on a chord to the side of drift shaft 24. The means by which portion 20 may be rendered selectively bendable in a single defined plane is not limited to the disclosed embodiments, but may include any of many well known means now known in the art or later discovered. The result is that burr 22 is selectively steerable by means of a bias or curve in the distal portion of catheter 18 which may be temporally or permanently provided.

Additional embodiments of the rotating-to-oscillating motion transmission are illustrated in FIGS. 4-9d which may be preferred over that described above. While many embodiments other than those described below are possible and while an oscillating motor is most preferred, it may not be practical to provide a directly oscillating drive which can effectively oscillated over the desired angular segment at 5000 to 8000 Hz. Most oscillating drives currently commercially available re limited to oscillation rates of 3000 Hz or less. Therefore, what is disclosed are three embodiments, an oscillating belt drive, an oscillating four cycle cam drive, and an oscillating geared drive. FIG. 4 is a simplified perspective view of transmission 50, which is comprised of a rotating drive shaft 52 fixed to segmental belt pulley 54 and segmental drive gear 60. Segmental belt pulley 54 is engaged with an endless belt 58 which in turn is engaged with positive driven pulley 56. In the illustration, pulleys 54 and 56 as well as belt 54 are shown as smooth, but it is contemplated that toothed pulleys and belts may be employed. Similarly, in the illustration of FIG. 4 segmental drive gear 60 and negative driven gear 62 are shown as smooth and frictionally engaged, but it is contemplated that gears 60 and 62 may be toothed.

It can readily be appreciated by viewing FIG. 4 that as shaft 52 is rotated, first segmental drive gear 60 is engaged with driven gear 62 which it rotates in a first sense, namely one opposite to the sense of rotation of shaft 52. As segmental drive gear 60 rotates, it comes to an orientation in which it no longer engages driven gear 62. At this point, segmental drive pulley 54 has been rotated until it is engaged with belt 58. Belt 58 is then driven thereby driving positive pulley 56 in the same sense of rotation as shaft 52. As a result, shaft 64 oscillates. The ratio of oscillation of shaft 64 to rotation of shaft 52 can be chosen according to the ratio of the drive gear and pulley 60, 54 to the driven gear and pulley 62, 56.

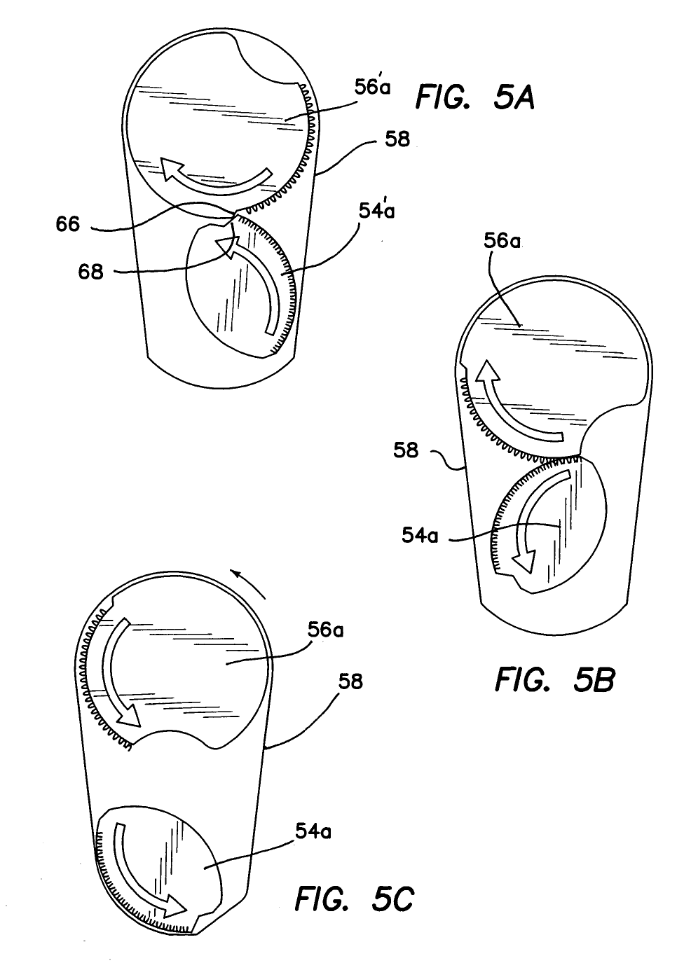

An alternative belt embodiment is illustrated in FIGS. 5a-5c. FIG. 5a is an end cut-away view of transmission 50 in which a portion of drive pulley-gear 54a and driven pulley-gear 56a is provided with teeth and in which both are segmental. Pulley-gear 54a is shown in FIG. 5a as being driven in a counterclockwise sense. Shoulder 68 of pulley-gear 54a comes into contact with shoulder 66 of driven pulley-gear 56a. This contact provides for positive drive of pulley-gear 56a and prevents damage to the gear teeth. At this point in the cycle, belt 58 is loose and is provided no motive power to driven pulley-gear 56a.

As shown in the end cut-away view of FIG. 5b pulley-gear 54a has driven pulley-gear 56a clockwise through their mutual toothed engagement. Belt 58 remains loose. Continued rotation of pulley-gear 54a brings it into engagement with belt 58 as shown in FIG. 5c, tensions belt 58 and causes it to rotate pulley-gear 56a in a counterclockwise sense. The cycle repeats with a return to the configuration of FIG. 5a. As a result, pulley-gear 56a will oscillate through an angular segment as determined by the relative segmental arc lengths of pulley-gears 56a and 54a. At the end of the reverse rotation cycle notice that there is a space on both pulley-gears 56a and 54a. Thus, if the oscillation gets out of phase, it will tend to be self correcting. The degree of belt tightness is determined by the shape of pulley-gears 56a and 54a. A rotational stop (not shown) is provided on the oscillating drive, as a safety measure. If one direction fails, the oscillator thus cannot go into a rotational mode. Also providing a spring on the oscillating shaft 64 with rest position at the start of negative rotation will tend to keep the oscillator in phase, help reverse direction of shaft 64 for oscillation, and thus increase efficiency. Transmission 50 may be stacked as shown in FIG. 4 or may be reduced to two elements as shown in FIGS. 5a-5c. In either case, it can be fit into a small space.

The most important design parameter of transmission 50 is the magnitude degrees of rotation for oscillation. By varying the relative size of the drive and driven elements, oscillations of 270 degrees and beyond are easily achieved. It is presently believed that oscillations in the range of about 30-60 degrees will be the most efficient, although any magnitude may be chosen according to the invention. Increasing the magnitude of occultation will increase cutting efficiency, but it also increases the tendency for the burr to jump, especially at lower oscillation frequencies. The best trade-off between stability and cutting efficiency as determined by the angular magnitude of occultation and frequency of oscillation can be empirically determined.

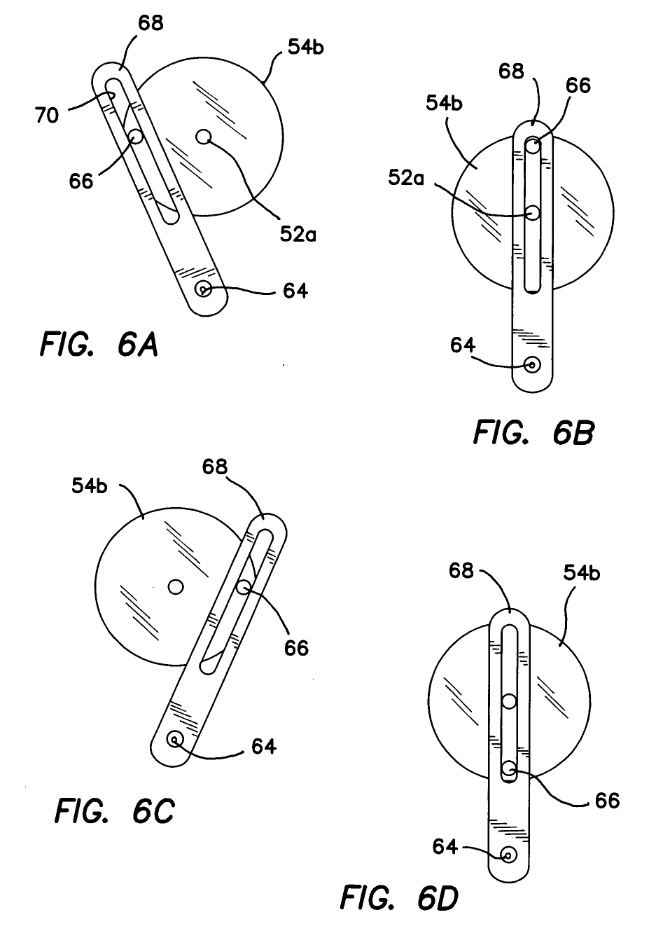

Another embodiment is shown in FIGS. 6a-6d which is a classical eccentric pin and wheel combination. A drive wheel 54b rotating about a center or shaft 52a carries a radially disposed eccentric pin 66. Pin 66 is slidingly disposed in slot 70 of a crank 68. As wheel 54b rotates, pin 66 oscillates in slot 70 as shown in the sequence of end views of FIGS. 6a-6d thereby oscillating crank 69 and its connected shaft 64. The angular degree of oscillation is chosen according to the size of wheel 54b and the position of slot 70 relative to shaft 64.

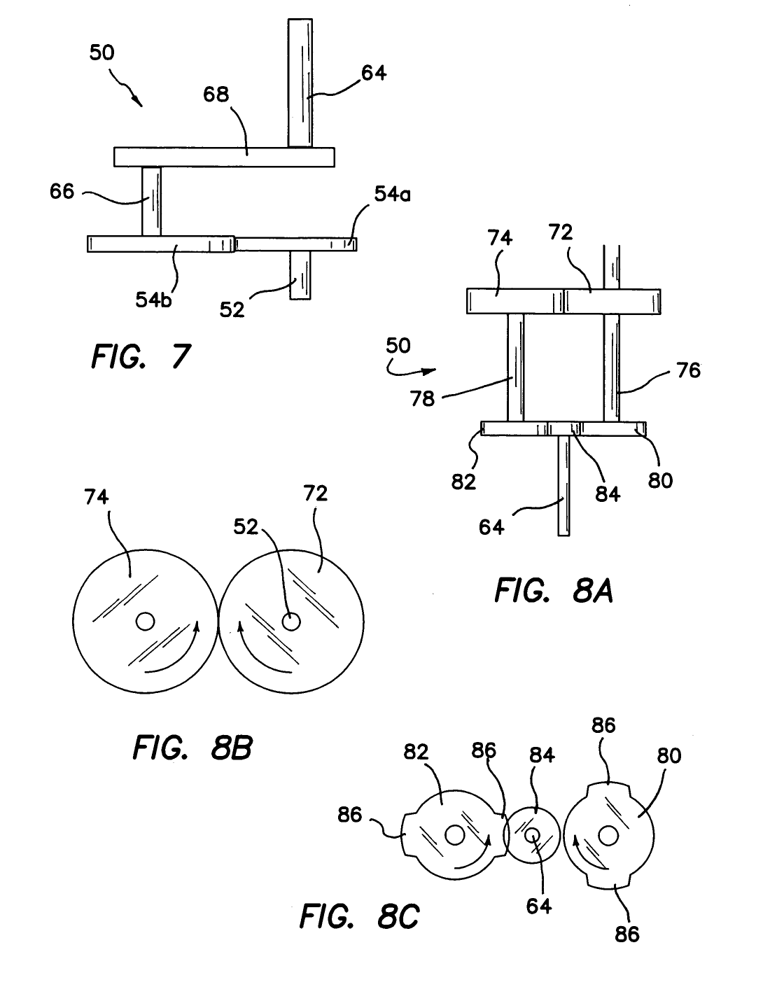

FIG. 7 is a diagrammatic top view of transmission 50 using the eccentric-crank combination of FIGS. 6a-6d. The axis of rotation 52a of wheel 54b is offset from the axis of oscillation 64 of crank 68. The axis of rotation and oscillation can be made coaxial by adding a drive gear 54a on drive shaft 52, which drive gear 54a engages gear 54b. The arc or angular magnitude of oscillation can easily be manipulated with such a set of gears 54a and 54b. Gears 54a and 54b are used to correct the offset between the axes of rotational drive and the oscillation output, and allows an easy means to choose the angular magnitude of rotation provided by choosing the gearing ratio between gears 54a and 54b.

A third embodiment for transmission 50 is diagrammatically illustrated in FIGS. 8a-8c. Rotational drive shaft 52 drives a forward gear 72, which is engaged with a reverse gear 74 so that gears 72 and 74 are counter-rotating as shown in end view in FIG. 8b. Forward gear 76 is connected to a first drive shaft 76 and reverse gear 74 is connected to a second drive shaft 78. Drive shaft 76 in turn is connected to a bibbed segmental gear 80, while drive shaft 78 is connected to a bibbed segmental gear 82 as depicted in end view in FIG. 8c. Each of the bibbed segmental gears 80 and 82 have diametrically opposing engagement or toothed portions 86 which intermittently engage output gear 84 connected to oscillating shaft 64. Segmental gears 80 and 82 are rotated 90 degrees with respect to each other so that they are one-quarter turn out of phase with each other. In FIG. 8c shaft 52 is rotating clockwise which rotates shaft 78 counterclockwise and shaft 76 clockwise. Hence, when gear 80 engages output gear 84, shaft 64 is rotated counterclockwise and when gear 82 engages output gear 84, shaft 64 is rotated clockwise in alternating fashion. The angular magnitude of the oscillation of shaft 64 can be varied according to the angular size of portions 86 on gears 80 and 82. Instead of being bilobed, gears 80 and 82 may be multiply lobed as well.

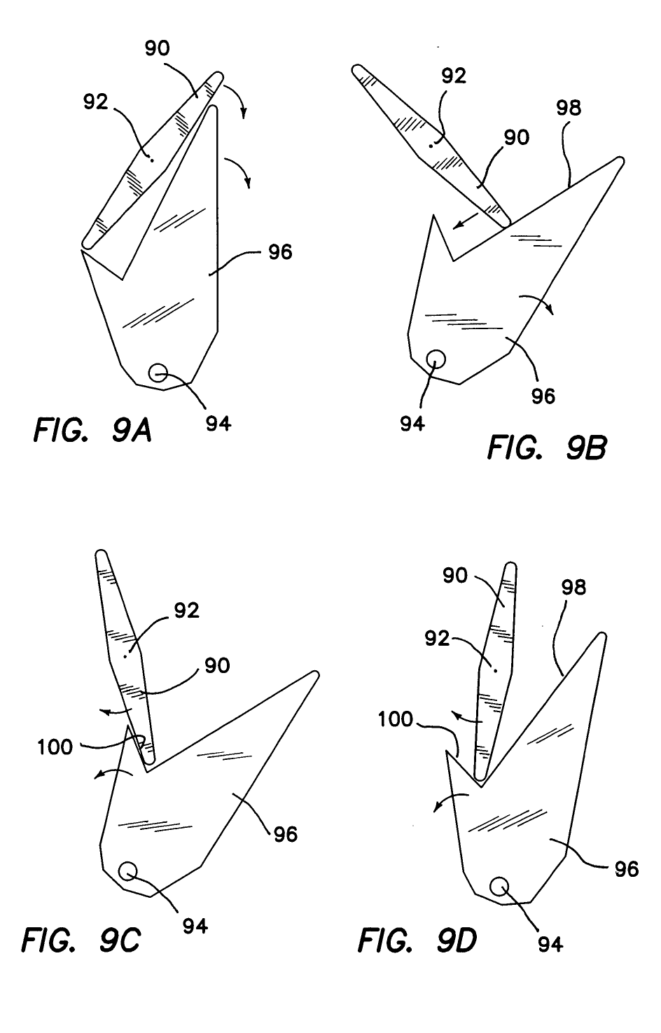

The geared embodiment of FIGS. 7a-7b may be replaced by a similarly structured cam combination, where the toothed section are replaced by equivalent cam surfaces. Yet another cam combination is shown in FIGS. 9a-9d. A symmetry vane-shaped cam 90 rotates on a rotating drive shaft 92 in a clockwise direction as shown in FIG. 9a. Cam 90 bears against surface 98 on cam 96 which rotates in a clockwise direction on an oscillating driven shaft 94. When cam 90 rotates to a position shown in FIG. 9c, it bears against surface 100 of cam 96, causing cam 96 to reverse and rotate in a counterclockwise direction. Counterclockwise rotation of cam 96 continues as cam 92 continues to rotate in a clockwise direction as shown in FIG. 9d bearing against surface 100. By the time cam 90 is just losing contact with surface 100 the opposing end of cam 90 is just approaching surface 98 of cam 96 as shown in FIG. 9a in preparation of again reversing direction of rotation of cam 96. Hence, cam 96 rotates through an arc twice for each rotation of cam 90. The camming combination of FIGS. 9a-9d can be coupled with a geared arrangement as described in connection with FIG. 7 to eliminate the offset in shafts 92 and 94 and to provide different gearing ratios between them.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

I claim:

1. A surgical apparatus for providing oscillating, high speed burring of tissue comprising:
a handpiece;
an oscillating burr;
an elongate arthroscopic catheter connected to and extending distally from the handpiece and terminating in a flexible or hinged variably curved portion which itself terminates with the oscillating burr;
a flexible drive shaft assembly coupled to the burr;
a motive source connected via the drive shaft assembly to the burr, which drive shaft assembly is axially disposed in the catheter; at least the distal portion of the drive shaft assembly being radially flexible to accommodate the flexibility of the flexible or hinged portion of the catheter.

2. The apparatus of claim 1 where the oscillating burr oscillates at a rate effective for cutting or abrading bone.

3. The apparatus of claim 1 where the oscillating burr oscillates at a rate of 5 kHz or higher.

4. The apparatus of claim 1 where the oscillating burr oscillates over a majority portion of a full circle.

5. The apparatus of claim 1 where the burr is unshielded and fully exposed in the operational theater, so that access to the burr is substantially unimpeded, so that cutting in virtually all directions is possible, so that cooling and clearing by fluid irrigation and fluid, and so that debris removal by suction can be performed without hindrance.

6. The apparatus of claim 1 where the burr cuts or abrades bone or hard matter, while leaving softer tissues substantially or entirely undamaged.

7. The apparatus of claim 1 further comprising a driving hub, a driven hub and a resilient spring coupled to the driven hub, the driving hub and driven hub being frictionally engagable with each other for a fraction of a revolution, and where the drive shaft assembly is connected to the burr through the driven hub.

8. The apparatus of claim 1 where the motive source is a source of rotary motion and the burr is oscillated by the drive shaft assembly, which converts the rotary motion into an oscillating motion.

9. The apparatus of claim 8 where the drive shaft assembly comprises a rotating driving shaft connected to the motive source, a rotationally fixed torsional spring, and a driven shaft frictionally coupled to the driving shaft and coupled the fixed to the torsional spring.

10. The apparatus of claim 9 where the driving shaft and the driven shaft are frictionally coupled by means of frictional engagement with each other in an overlapping portions are telescopically disposed into or over each other.

11. The apparatus of claim 8 where the drive shaft assembly comprises a segmental gear-pulley combination with a belt.

12. The apparatus of claim 8 where the drive shaft assembly comprises an eccentric pin-crank combination.

13. The apparatus of claim 8 where the drive shaft assembly comprises a bibbed counter-rotating gear combination.

14. A method of oscillating, a high speed surgical burr comprising:
providing a motive source;
connecting the motive source via a drive shaft assembly to the burr; and
oscillating the burr at a oscillatory rate effective for cutting or abrading bone over a portion of a full circle so that the burr cuts or abrades bone or hard matter, while leaving softer tissues substantially or entirely undamaged.

15. The method of claim 14 where oscillating the burr over a portion of a full circle oscillates the burr over a majority portion of a full circle.

16. The method of claim 14 where the oscillatory rate effective for cutting or abrading bone is at 10 kHz or higher.

17. The method of claim 14 further comprising providing a burr which is unshielded and fully exposed in the operational theater, and cutting with the burr virtually all directions without substantial impediment.

18. The method of claim 14 further comprising providing a burr which is unshielded and fully exposed in the operational theater, cooling and clearing the burr by fluid irrigation and fluid, and removing debris by suction without hindrance.

19. The method of claim 14 further comprising coupling the drive shaft assembly to the burr by a resiliently biased slip clutch.

20. The method of claim 14 where the motive source is a source of rotary motion and further comprising oscillating the burr by means of the drive shaft assembly, which converts the rotary motion into an oscillating motion.

21. The method of claim 20 further comprising rotating the driving shaft of the drive shaft assembly which driving shaft is connected to the motive source, partially rotating a driven shaft of the drive shaft assembly in a first sense of rotation by means of frictional coupling of the driven shaft to the driving shaft, and partially rotating the driven shaft in a second sense of rotation opposite to the first sense of rotation by means of a rotationally fixed torsional spring coupled to the driven shaft, so that the driven shaft oscillates as the driving shaft rotates.

22. The method of claim 20 where oscillating the burr by means of the drive shaft assembly comprises frictionally coupling the driving shaft and the driven shaft by means of frictional engagement with each other in an overlapping portion telescopically disposed into or over each other.

23. The method of claim 20 where oscillating the burr by means of the drive shaft assembly comprises oscillating the burr by means of a segmental gear-pulley combination with a belt.

24. The method of claim 20 where oscillating the burr by means of the drive shaft assembly comprises oscillating the burr by means of an eccentric pin-crank combination.

25. The method of claim 20 where oscillating the burr by means of the drive shaft assembly comprises oscillating the burr by means of a bibbed counter-rotating gear combination.

26. A method of oscillating, a high speed surgical burr comprising:
providing a motive source;
connecting the motive source via a drive shaft assembly to the burr; and
oscillating the burr at a oscillatory rate at 10 kHz or higher over a majority portion of a full circle so that the burr cuts or abrades bone or hard matter, while leaving softer tissues substantially or entirely undamaged with a burr which is unshielded and fully exposed in the operational theater, which is capable cutting with the burr virtually all directions without substantial impediment, which is cooled and cleared by fluid irrigation and fluid, and from which debris is removed by suction without hindrance.
